(19) United States
(12) Patent Application Publication
(10) Pub. No.: US 2017/0080117 A1
(43) Pub. Date: Mar. 23, 2017

(54) SYSTEM AND METHOD FOR INACTIVATING PATHOGENS USING VISIBLE LIGHT AND/OR UV LIGHT

(71) Applicant: LiteProducts LLC, Weston, CT (US)

(72) Inventor: Peter GORDON, Weston, CT (US)

(21) Appl. No.: 15/366,687

(22) Filed: Dec. 1, 2016

Related U.S. Application Data
(63) Continuation-in-part of application No. 14/801,293, filed on Jul. 16, 2015.
(60) Provisional application No. 62/025,070, filed on Jul. 16, 2014.

(51) Int. Cl. A61L 2/24; A61L 2/10; A61L 2/08

(52) U.S. Cl. CPC A61L 2/24; A61L 2/084; A61L 2/10

(57) ABSTRACT

A system for inactivation of pathogens on a surface may include a first light source that emits first light having a peak wavelength in a range of 100 nm to 500 nm; a second light source that emits second light that is visible light; control electronics configured to control a first power state of the first light source and a second power state of the second light source. The first power state and the second power state may be independently controlled. The system may be configured such that an illumination area of the first light and the second light is limited to the inactivation area.

### CROSS REFERENCE TO RELATED APPLICATIONS

The present application is a continuation-in-part application of U.S. patent application Ser. No. 14/801,293 filed on Jul. 16, 2015, which is incorporated herein by reference in its entirety. This application also claims the benefit of priority under 35 U.S.C. §119(e) based on U.S. Provisional Application Ser. No. 62/025,070 filed on Jul. 16, 2014, the entire content of which is also incorporated herein by reference.

### TECHNICAL FIELD

The present application relates to the inactivation of pathogens using visible light and/or UV light.

### BACKGROUND

Infectious diseases are caused by various pathogens: vegetated bacteria, bacterial spores, virions, fungus, etc. Once upon or within the body they replicate and can cause an infection and illness, sometimes resulting in death. Pathogens act by entering the body through openings, by way of contaminated food, fomites, and aerosolized pathogens in air or on dust, human contact with pathogen contaminated surfaces, or human-to-human contact. The contaminated hands of healthcare workers in hospitals and clinics are a significant vehicle for transmission of infectious pathogens to patients. Hands are invariably contaminated by contact with surfaces that are typically contaminated; usually unavoidably. This is especially common in hospitals. As a result in the US of order 7% of patients acquire infectious diseases as a result of a hospital stay and approximately 100,000 die annually. Worldwide infection statistics are equally dismal.

The most important sources of hand contamination in hospitals are patients, contaminated surfaces, contaminated clothing worn by healthcare workers, instruments such as stethoscopes and air. Visitors are another source of pathogen contamination. The contamination problem is not confined to hospitals; contaminated hands are also capable of transferring pathogens to food, food handling equipment, and to laboratory equipment.

In a hospital or other health care environment, surgeons, physicians, nurses, other health care workers, and visitors are significant causative factors in transmission of infectious pathogens to patients and from patient to patient by virtue of inadequate attention to or omission of use or unavailability of technology for proper hand sanitation. Sanitation of a surface such as the hand is strictly and technically defined in the context of infection control as a reduction of pathogens of any given type per unit area of the surface to 10-4 times the value before sanitation; technically, sanitation to a level of −4 log 10 reduction or 99.99% inactivation of surface pathogens is also referred to as disinfection.

The traditional method of achieving pathogen reduction is hand washing with regular or anti-microbial soap and drying with sterilized towels. Only prolonged hand washing achieves technical sanitation and it does so by removing transient pathogens from the surface of the hands. It does not kill pathogens. Instead of merely removing pathogens, it would be desirable to inactivate the pathogens. In this context, inactivation means making the pathogen incapable of multiplying so it cannot cause infection. In the last 20 years the application of alcohol formulations, (‘rubs’), followed by a short air drying period taking a total of 30 seconds has become a common pathogen inactivation process for bare hands, although alcohol rubs do not quite achieve technical sanitation with respect to vegetated bacteria, does not inactivate certain virus, and it does not inactivate any endospores (“spores”).

Typical washing of hands and forearms is capable of removing a fraction of the transient pathogens of all kinds on or near the skin surface, whereas alcohol rubs as noted are ineffective on spores such as C. difficile, which annually kill 21,000 hospital patients. Each technique has inadequacies such as: 1) elimination or reduction to 10-4 of the original number of active pathogens, technical sanitation, is seldom accomplished or assured; 2) the conventional techniques do not uniformly cover 100% of the area supposedly sanitized; and 3) the conventional techniques are not always possible or convenient to implement for multiple reasons. The result is a variable rate of disinfection compliance between patient visits, usually less than 50%, and there is uncertainty in achieving technical sanitation when it is implemented.

Extended application time improves the protection. For example, surgeons scrub their hands for many minutes to improve the percentage of pathogens removed. Nurses and other healthcare workers with far less time available wash their hands for about 60 seconds, many times daily, and as a result, cause their hands become painfully sore and chapped; thereby making it difficult to use the hand wash technique consistently.

Thus, due to these unpleasant side-effects, bare hand sanitation is inconsistently applied. It is estimated that bare hand sanitation is practiced less than 40% of the time between patient visits, and generally not at all during the patient visit. The classic explanations for non-compliance are: 1) inadequate time given the busy schedules of the healthcare workers, and 2) hand irritation. Although requiring less time and being less irritating, the use of alcohol rub does not significantly improve the compliance rate.

Moreover, wearing exam or surgical gloves does not mitigate these problems. As health care professionals go from patient to patient, they transport pathogens on the surfaces of the gloves just as readily as they do on bare hands. Glove surfaces are not sanitized since the practical purpose of wearing gloves is to protect the wearer from the patient. The contaminated surfaces do not protect the patient. Since surfaces in the hospital room are invariably contaminated, the surface of exam gloved hands quickly becomes contaminated by anything they touch. One touch of any surface by the hand contaminates the surface of the hand. All the effort at sanitation between patient visits can be lost by a single touch by the hand of any surface, including clothing, instruments, data input devices, or by settling of aerosols or fomites containing pathogens drifting in the air. The contaminated hand, bare or gloved, is a major vehicle for transmission of pathogens to the patient and is believed to be the primary vehicle for spread of hospital acquired infections. Furthermore, it is generally understood that the purpose of the gloves is to protect the healthcare worker from the patient, not the patient from the healthcare worker. Gloves are not typically washed. Hence, the use of gloves has little or no impact on the patient infection problem and provides no protection for the patient. Surgical gloves are nominally sterile but sterility is not guaranteed.

The World Health Organization, WHO, maintains that the bare hand should be sanitized at bedside immediately before the patient is to be touched. Currently there is not a practical or viable way to implement that plan, and it also does not deal with the issue of glove contamination. Ultimately current hand sanitation technologies; i.e., hand washing, alcohol rub, and use of gloves; are impractical and inadequate.

Bare hands are also a major element in the spread of infection in schools. Controlled studies have demonstrated that the student absentee rate is reduced by 50% with proper hand washing just before lunch. Infected students miss class time and carry illnesses home. Improper hand sanitation in the school environment is a detriment to the absent students who miss class time. and a problem for family members who become ill from infections brought home by their children at school.

Washing hands is typically not practiced as frequently as desired or in an adequate manner. Moreover, in many developing countries, the sanitary and hygienic conditions at schools are often very poor, and can be characterized by the absence of properly functioning or existing water supply for sanitation or hand washing facilities.

Sanitary hands in take-out food service or restaurant settings are similarly critical to prevent the spread of disease. The FDA reports that poor personal hygiene in a food service environment is a critical area that needs immediate attention and sets the following requirements with respect to personal hygiene: ‘Proper and adequate hand-washing, prevention of hand contamination, good hygienic practices, and a hand-washing facility that is convenient and accessible, with cleanser/drying devices.’

A summary of several studies and initiatives concerning hand-hygiene can be found in an article by Kelly M. Pyrek, entitled “Hand Hygiene: New Initiatives on the Domestic and Global Fronts,” published Jun. 1, 2006, and available at a web site maintained by Infection Control Today (ICT).

Thus, there is clearly a need for an effective device and method of pathogen inactivation that can be conveniently implemented without the drawbacks associated with hand washing or alcohol rubs.

Recent research has raised the possibility of a technique for sanitation of room surface using visible light wavelengths (see, for example, USPGP 2015/0182646 and “Bactericidal Effects of 405 nm Light Exposure Demonstrated by Inactivation of Escherichia, Salmonella, Shigella, Listeria, and Mycobacterium Species in Liquid Suspensions and on Exposed Surfaces,” Scientific World Journal, published online Apr. 1, 2012). The most active wavelength band was in the blue part of the visible spectrum with peak activity at a wavelength of approximately 405 nm. The illumination source was LEDs known as High Intensity Narrow Spectrum (HINS) light. It is claimed that absorption of HINS-light wavelengths by intracellular molecules induces production of reactive oxygen species within molecules and this causes inactivation of pathogens. It is harmless to humans because the illumination is visible light.

In these previous experiments, however, one or more LED light sources located in ceiling fixtures illuminate the entire room. Over a period of order 24 hours it reduced bacterial counts by a factor of less than ten. Given the amount of time required and the amount of bacterial inactivation, these devices and techniques would be inadequate for pathogen inactivation in a faster paced, higher traffic, clinical or commercial setting where more rapid results are required.

Therefore, there is a need in the art for a devices and methods of pathogen inactivation using light that would be effective on a much shorter scale of time, and that inactivates a greater number of pathogens.

### SUMMARY

A system for inactivation of pathogens within an inactivation area on a surface may include a first light source that emits first light having a peak wavelength in a range of 400 nm to 500 nm; a second light source that second emits light having a peak wavelength in a range of 185 nm to 400 nm; and control electronics configured to control a first power state of the first light source and a second power state of the second light source. The first power state and the second power state may be independently controlled. An illumination area of the first light and an illumination area of the second light may be limited to the inactivation area.

A method of inactivating pathogens within an inactivation area on a surface may include providing a system include a first light source that emits first light having a peak wavelength in a range of 400 nm to 500 nm, a second light source that emits second light having a peak wavelength in a range of 185 nm to 400 nm, and a light guide structured to direct the first light and the second light to the inactivation area; setting a first power state of the first light source to an on state; aiming the light guide so that the first light illuminates the inactivation area; and setting the second power state of the second light source to an on state. An illumination area of the first light and an illumination area of the second light may be limited to the inactivation area.

A system for inactivation of pathogens on a surface may include a first light source that emits first light having a peak wavelength in a range of 185 nm to 500 nm; a second light source that emits second light that is visible light; and control electronics configured to control a first power state of the first light source and a second power state of the second light source. The first power state and the second power state may be independently controlled. The second light source may be configured to emit light in a predetermined pattern that indicates an inactivation area to be illuminated. The system may configured such that an illumination area of the first light is limited to the inactivation area.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a schematic front view of an embodiment of a device for inactivation of pathogens.

FIG. 2 is a schematic front view of an embodiment of a device for inactivation of pathogens.

FIG. 3 is a cross-sectional schematic side view of an embodiment of a device for inactivation of pathogens.

FIG. 4 is a cross-sectional schematic side view of an embodiment of a device for inactivation of pathogens.

FIG. 5 is a schematic front view of an embodiment of a device for inactivation of pathogens.

FIG. 6 is a schematic front view of an embodiment of a device for inactivation of pathogens.

FIG. 7 is a cross-sectional schematic side view of an embodiment of a device for inactivation of pathogens.

FIG. 8 is a perspective view of an embodiment of a device for inactivation of pathogens.

FIG. 9 is a perspective view of an embodiment of a device for inactivation of pathogens.

FIG. 10 is a side view of an embodiment of a device for inactivation of pathogens.

FIG. 11 is a schematic perspective view showing a possible use of an embodiment of a device for inactivation of pathogens.

FIG. 12 is a perspective view showing a possible mounting of an embodiment of a device for inactivation of pathogens.

FIG. 13 is a side view showing a possible mounting of an embodiment of a device for inactivation of pathogens.

FIG. 14 is a perspective view of an embodiment of a device for inactivation of pathogens.

FIG. 15 is a perspective view showing an embodiment of hand placement verification for use in an embodiment of a device for inactivation of pathogens.

FIG. 16 shows graphs of the output of an embodiment of hand placement verification for use in an embodiment of a device for inactivation of pathogens.

FIG. 17 shows a schematic view of an embodiment of a handheld device for inactivation of pathogens.

FIG. 18 is a top planar view of an embodiment of a handheld device for inactivation of pathogens.

FIG. 19 is a perspective view of an embodiment of a device for inactivation of pathogens on a surface.

FIG. 20 is a perspective view of an embodiment of a device for inactivation of pathogens on a surface.

FIG. 21 is a perspective view of one possible use of an embodiment of devices for inactivation of pathogens on a surface.

FIG. 22 is a view an embodiment of a system for inactivation of pathogens.

FIG. 23 is a view an embodiment of a system for inactivation of pathogens.

FIG. 24 is a perspective view of an embodiment of a system for inactivation of pathogens for use with a food service table.

FIG. 25 is a view of an embodiment of a system for inactivation of pathogens for use with a public terminal.

FIG. 26 is a view of an embodiment of a system for inactivation of pathogens.

FIG. 27 is a view of an embodiment of a system for inactivation of pathogens.

FIG. 28 is a flowchart of an embodiment of a method for inactivation of pathogens.

FIG. 29 is a flowchart of an embodiment of a method for inactivation of pathogens.

FIG. 30 is a flowchart of an embodiment of a method for inactivation of pathogens.

FIG. 31 is a view of an embodiment of a system for inactivation of pathogens.

FIG. 32 is a view of an embodiment of a system for inactivation of pathogens.

FIG. 33 is a view of an embodiment of an inactivation area and an aiming pattern.

FIG. 34 is a view of an embodiment of an inactivation area and an aiming pattern.

FIG. 35 is a view of an embodiment of an inactivation area and an aiming pattern.

FIG. 36 is a view of an embodiment of an inactivation area and an aiming pattern.

FIG. 37 is a view of an embodiment of an inactivation area and an aiming pattern.

FIG. 38 is a view of an embodiment of an inactivation area and an aiming pattern.

### DETAILED DESCRIPTION OF EMBODIMENTS

FIG. 1 shows a front view of at least one embodiment of a device for inactivation of pathogens on an object. As seen in FIG. 1, the device may include a main body 100 defining an internal space 110. A first light source 120 may be provided on a first internal surface 130 of main body 100. Internal space 110 accommodates the object 140. First light source 120 may emit light having a wavelength in the range of 400 nm to 500 nm.

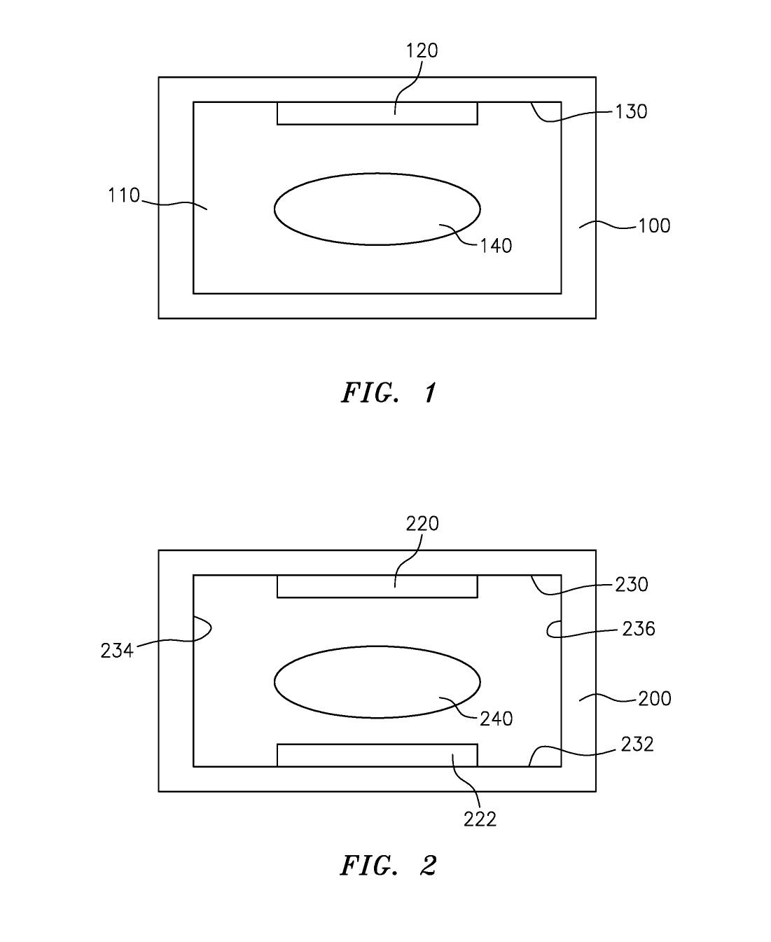

FIG. 2 shows a front view of at least another embodiment of a device for inactivation of pathogens on an object. In the embodiment of FIG. 2, a second light source 222 may be provided on a second internal surface 232 of main body 100. In present FIG. 2, second internal surface 232 and second light source 222 are opposite of first internal surface 220 and first light source 222. The first light source 220 and second light source 222 may emit light having a wavelength in the range of 400 nm to 500 nm. Additionally, in at least an embodiment, internal surfaces 230, 232, 234, 236 of main body 200 are reflective.

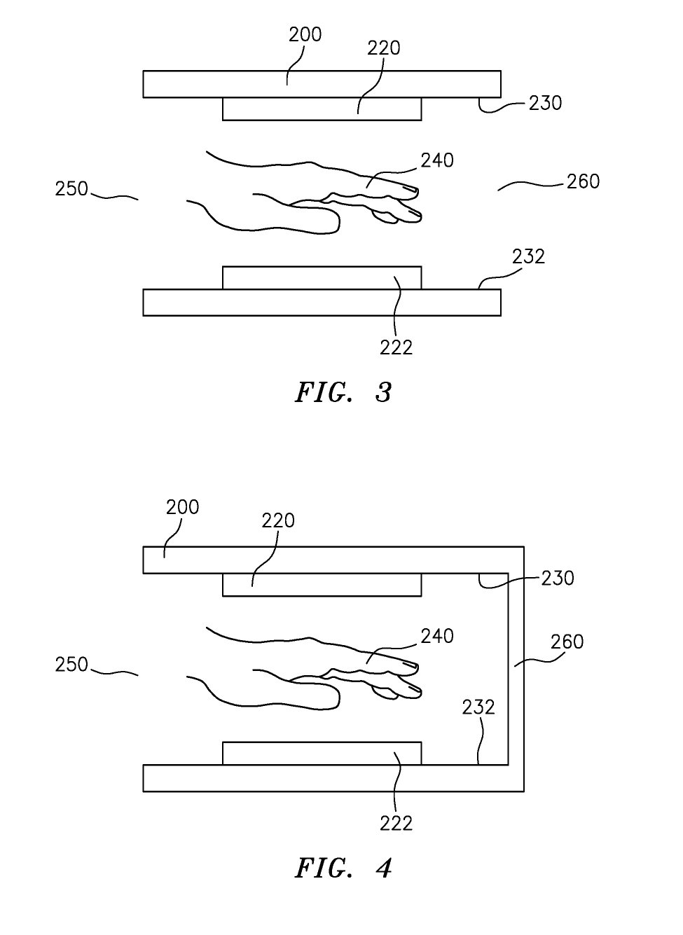

FIG. 3 shows a side cross-section view of the embodiment shown in FIG. 2. In the embodiment shown in FIG. 3, the main body 200 is a cylinder, column, or box shape that is open on a first end 250 and a second end 260. FIG. 4 shows another embodiment of a side cross-section view of the embodiment shown in FIG. 2. In the embodiment shown in FIG. 4, the main body 200 is open on a first end 250 and closed on a second end 260.

In FIGS. 3 and 4, the object 240 is a hand. However, it will be understood that the device is not limited to inactivating pathogens on only hands. For example, any suitable object such as instruments, utensils, trays, dishes, glassware, lab equipment, or any other object that fits inside the device can be subject to pathogen inactivation.

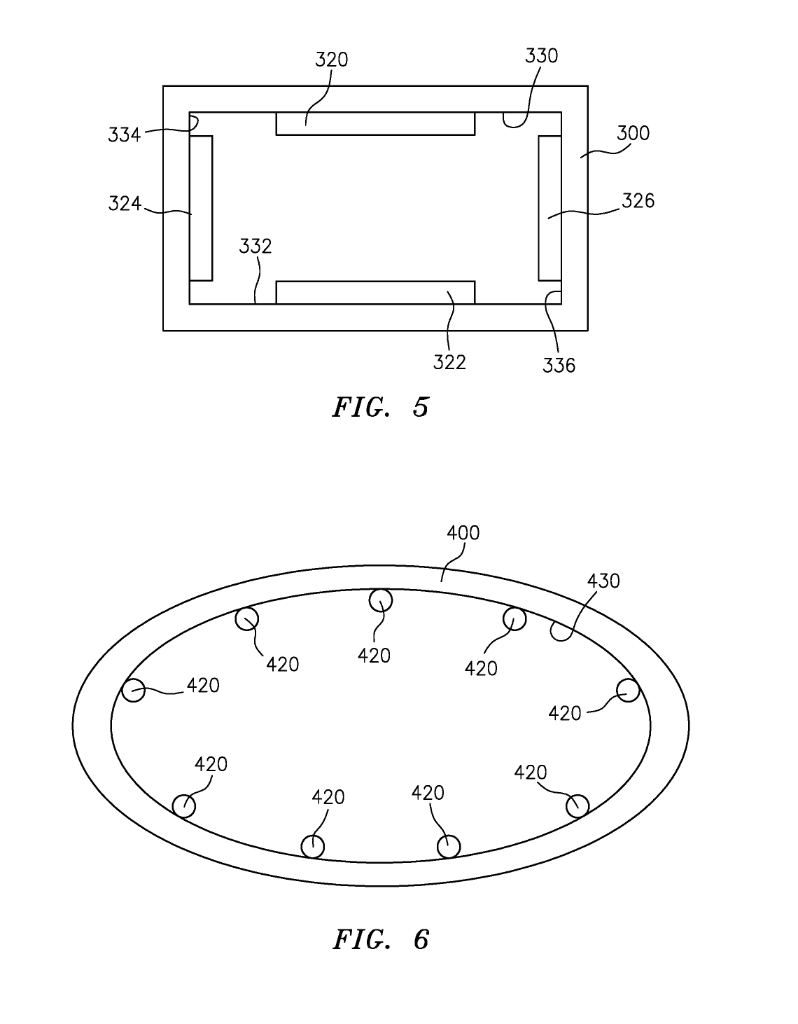

FIG. 5 illustrates another embodiment of a device for inactivation of pathogens on an object. In FIG. 5, there is a plurality of light sources 320, 322, 324, 326 provided on internals surfaces 330, 332, 334, 336 of main body 300. Light sources 320, 322, 324, 326 emit light having a wavelength of 400 nm to 500 nm, internal surfaces 330, 332, 334, 336 are reflective.

It will also be understood that the device is not limited to a rectangular or cubic shape. For example, FIG. 6 shows an embodiment in which the main body 400 has an elliptical cross section, and a plurality of light sources 420 provided on an internal surface 430 of main body 400. The cross section of the main body of the device can have any suitable shape, such as rectangle, ellipse, circle, or other polygon or curved shape.

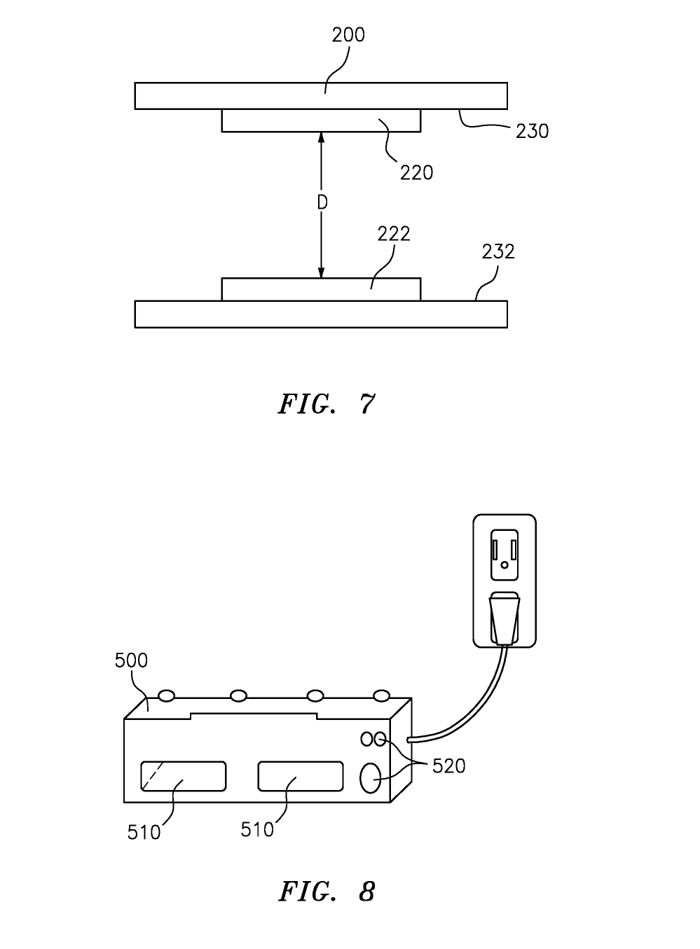

In FIG. 7, D represents the distance between first light source 220 and second light source 222. In at least one embodiment, distance D is 20 cm or less. In another embodiment, distance D is 10 cm or less. In yet another embodiment, distance D is 5 cm or less.

Regarding the light sources described above, there are a number of different possible options to use as the light source. For example, any of the light sources discussed above can comprise an array of LEDs. As just one possible example, the LED array may be formed from InGaN LEDs, which emit light in the range of 400-500 nm. However, the device is not limited to InGaN LEDs, as any LED that emits light in the range of 400-500 nm can be used. In addition to LEDs, it is possible to also use cold cathode lamps or low pressure lamps that emit light in the range of 400-500 nm.

In the description above, it has been noted that the various light sources emit light having a wavelength in the range of 400-500 nm. It will be understood that in addition to this range, at least an embodiment of the device will have light sources that emit light having a wavelength in the range of 400-410 nm. It will be further understood that at least an embodiment of the device will have light sources that emit light having a wavelength in the range of 404-406 nm. It will be further understood that at least an embodiment of the device will have light sources that emit light having a wavelength of approximately 405 nm.

Additionally, in at least an embodiment, the light source will emit light only within the specified range. For example, in at least an embodiment of the device, the light sources emit only light having a wavelength in the range of 400-500 nm. Additionally, at least an embodiment of the device will have light sources that emit only light having a wavelength in the range of 400-410 nm. It will be further understood that at least an embodiment of the device will have light sources that emit only light having a wavelength in the range of 404-406 nm. It will be further understood that at least an embodiment of the device will have light sources that emit only light having a wavelength of approximately 405 nm.

The effectiveness of the device in inactivating pathogens on the object depends on the dose of light irradiated on the object. For example, a total dose of 30 J/cm2 is adequate for 10-4 (i.e. 99.99%) inactivation of MRSA pathogens. This dose would be sufficient to achieve the standard of sanitation, which is defined as inactivation of 99.99% of pathogens. This dose could be achieved in 30 seconds of time when the irradiance of the object is 1 W/cm2. The time of necessary exposure can be varied by changing the irradiance of the object. For example, 30 seconds exposure may be inconvenient in some applications. However, if the irradiance of the object is increased to 10 W/cm2, then the total required exposure time will only be 3 seconds to achieve the dose adequate for 99.99% inactivation. The irradiance of the object depends on the power of the light source and the area over which the light is directed. For example, if the target field for the object is 1000 cm2, then the light sources would need to have a power of 1000 watts to achieve 1 W/cm2 irradiance.

FIGS. 8-14 show various embodiments of a device for inactivation of pathogens. For example, FIG. 8 shows a device 500 that includes two slots 510 through which hands or other objects can be inserted. Device 500 may include interface 520. Interface 520 may include indicator lights that can indicate when an object is inserted into the device and when a sufficient time for the desired inactivation has passed. Interface 520 may also include controls to allow a user to modify the power output of the device, desired exposure time, change modes, or perform other suitable functions.

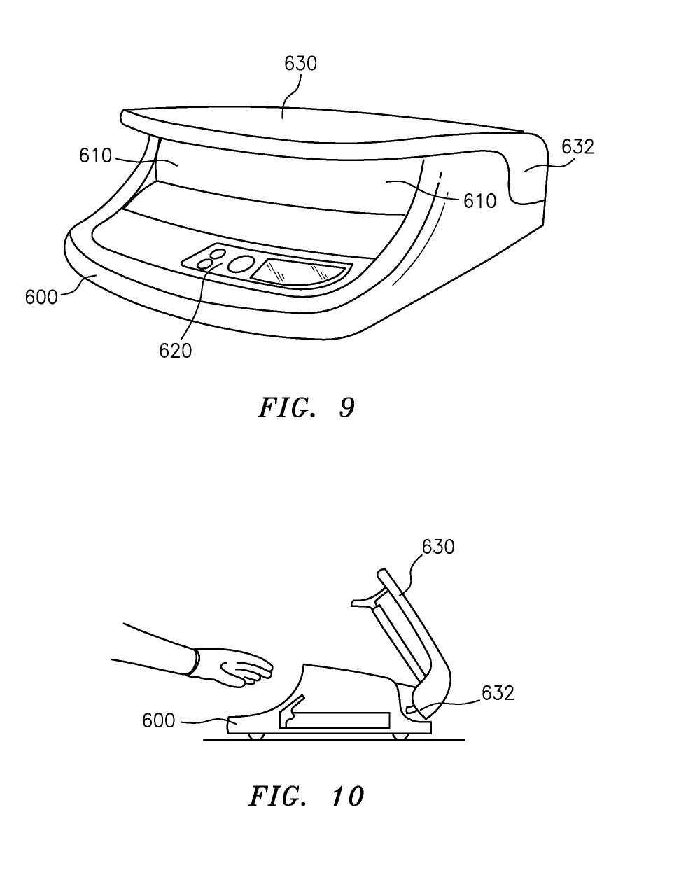

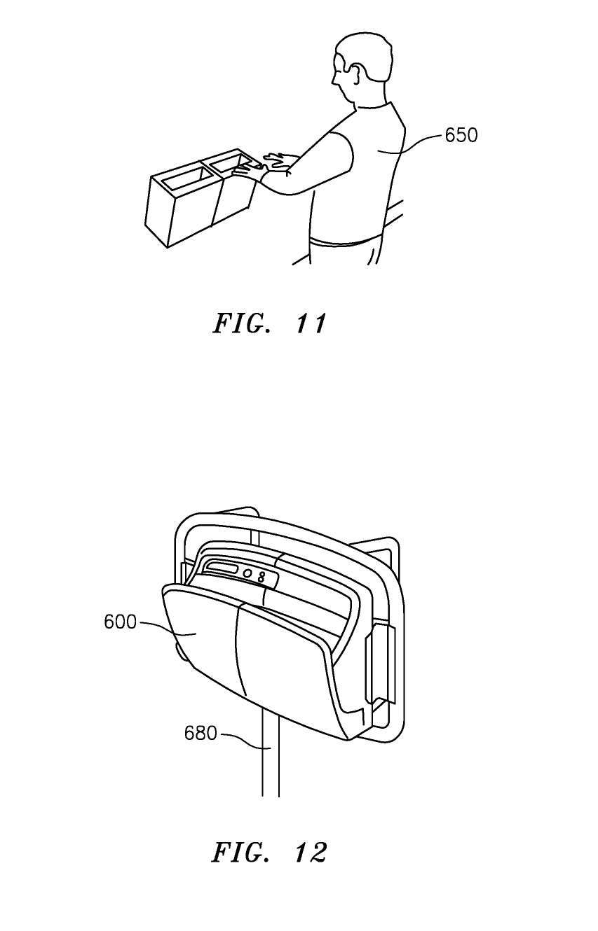

FIGS. 9-10 show another embodiment of a device 600 for inactivation of pathogens. In device 600, the slots 610 are placed side by side in a horizontal arrangement. This may allow for the sharing of some components between the two slots 610. Device 600 may further include an interface 620 that may include indicator lights, controls, and/or digital displays. As further seen in FIGS. 9-10, device 600 may have a top panel 630 that can be opened via hinge 632 to allow for easy cleaning and maintenance of device 600.

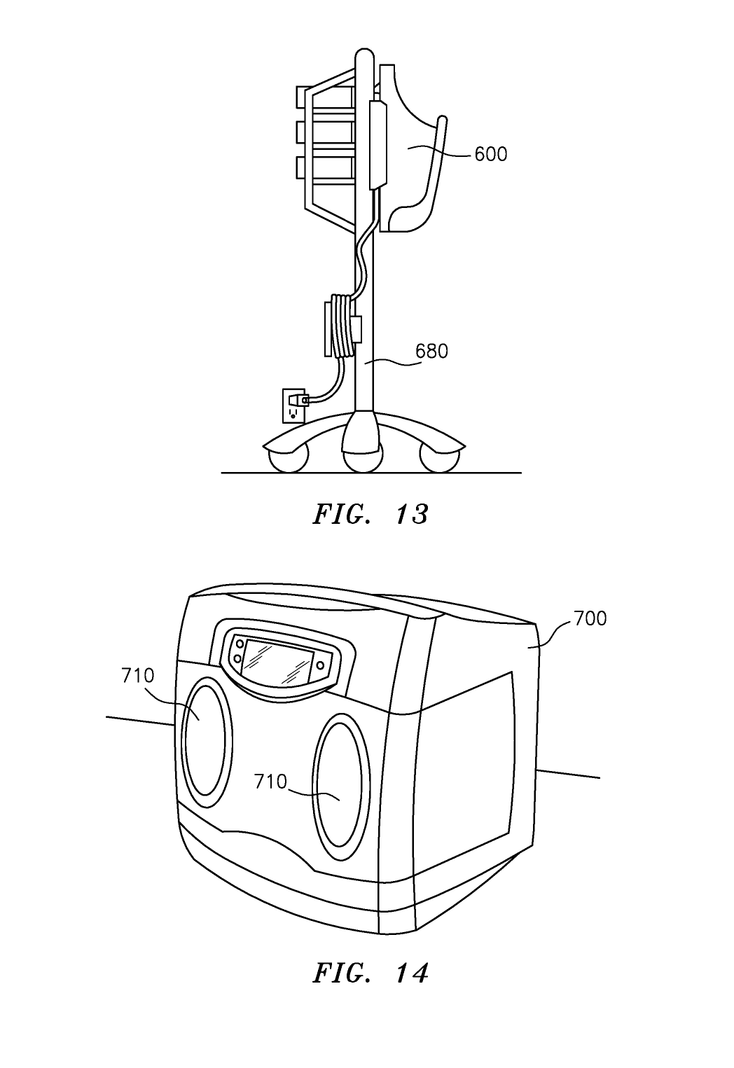

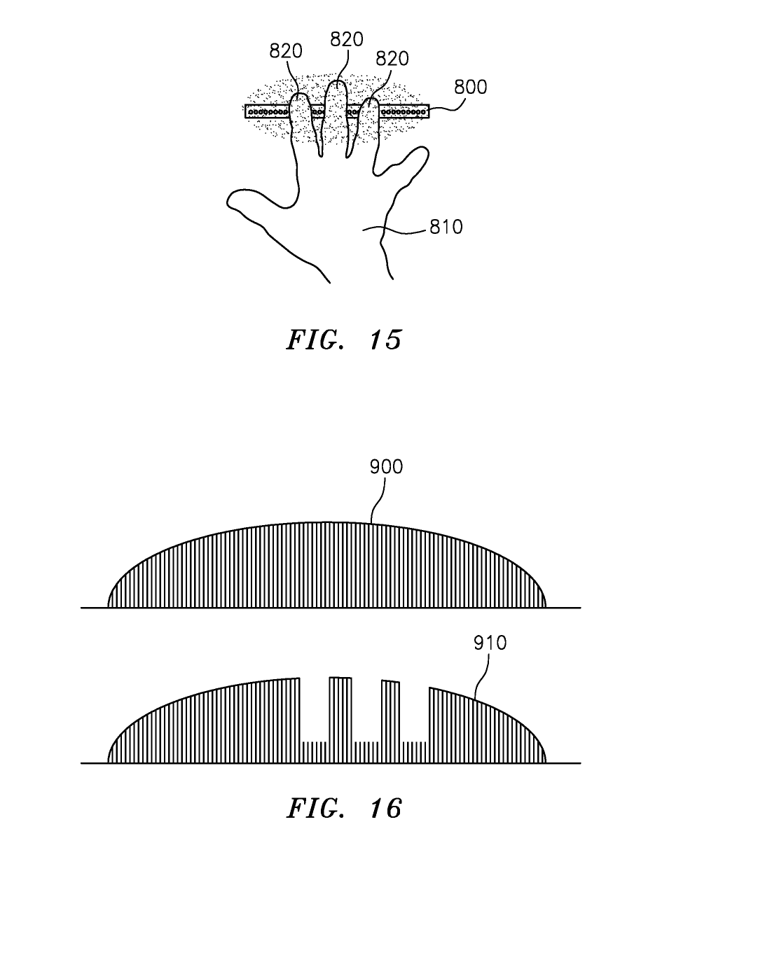

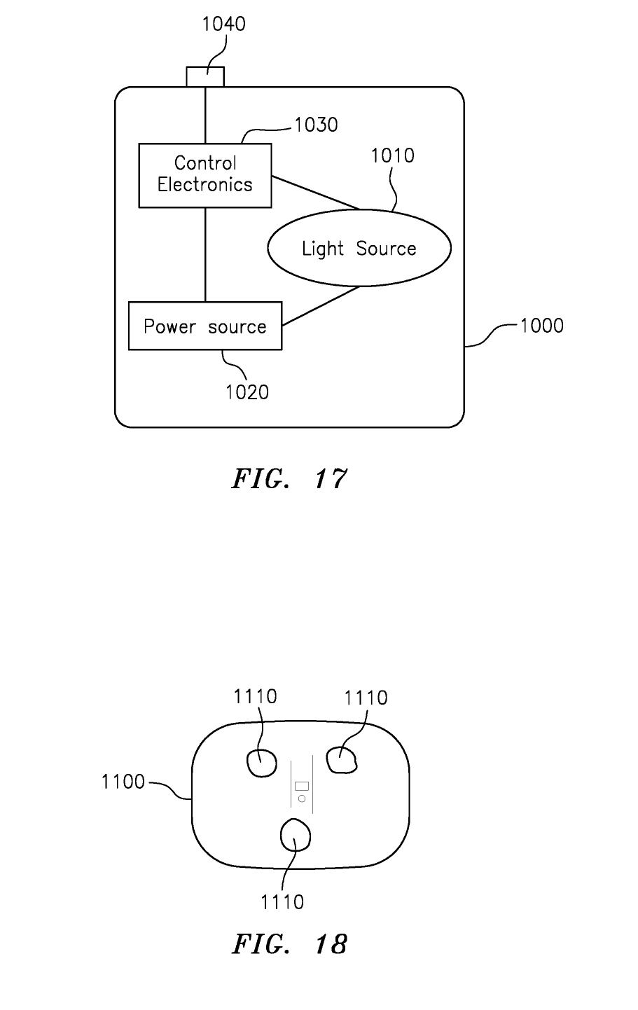

FIG. 11 shows a schematic view of how a device 600 can be arranged vertically for a smaller footprint, thereby saving space. A vertical arrangement of device 600 may be more comfortable for a variety of users 650. FIG. 12 shows how a device 600 can be mounted vertically on a pole mount 680. FIG. 13 shows that the pole mount 680 may be wheeled so that the device can be easily and conveniently moved to wherever pathogen inactivation is needed. FIG. 14 shows another embodiment of a device 700 for inactivation of pathogens in which the slots 710 are arranged vertically instead of horizontally. The vertical arrangement of slots 710 may be more comfortable for certain users in certain configurations.

As noted above, one drawback to conventional pathogen inactivation regimes using soap or other chemicals is that it is difficult to insure a consistent and uniform level of pathogen inactivation. With a device for inactivation of pathogens using visible light, as long as a users hands are presented so as to allow the light to reach all surfaces of the hands, it is possible to achieve much more consistent levels of pathogen inactivation. To insure that users are placing their hands properly, a device may include a sensor 800 such as a photodiode array at an appropriate position inside the device, as seen in FIG. 15. When a user's hand 810 is positioned properly and fingers 820 are spread, it can be seen that portions of the sensor will be shaded by the fingers. FIG. 16 shows a graph 900 showing a projected output of the sensor 800 with no hand present. When a hand is inserted, perhaps triggering a movement sensor to initiate the sanitation episode, and fingers are properly spread, the output of the sensor 800 will have a predictable variation in its shape, as shown in graph 910. By using analyzing the output of the sensor 800, a processor can determine whether the hands are in a proper position. Proper positioning can be acknowledged to the user by using an indicator light, a display, an audio cue, or other suitable sensory stimulus.

Thus, it will be understood that one advantage of the device over conventional methods of surface pathogen in activation is that the light can be delivered consistently over 100% of a user's hand with no required input from the user. This is a marked advantage over soaps or alcohol rubs, where the uniformity of exposure depends on the diligence of the user, and even there areas such as under fingernails or in cracks of skin may be missed.

Additionally, it is important to note that the visible light emitted by these devices does not damage or dry out the skin as water, soaps, and alcohol rubs are known to do. Therefore, because the hands would not be subjected to as much discomfort and damage, health care workers would be more likely to comply with hand sanitation protocols, thereby reducing infection rates.

Additionally, these benefits are not limited to the health care industry. Embodiments of the device could be used in commercial settings such as restaurants, food preparation, veterinary, animal husbandry, laboratories, public restrooms, day care centers, educational facilities, etc. Not only would these uses reduce contamination and infection, but they would also be environmentally friendly by reducing water use, chemicals from soap use, and paper towel waste.

FIG. 17 shows a schematic of an alternative embodiment in which the device is a portable, handheld device that can be carried on a person and used for pathogen inactivation whenever desired. For example, the device may include a main body 1000, a light source 1010, a power source 1020 such as a rechargeable battery or other suitable power source, control electronics 1030, and user interface 1040. The light source 1010 emits light having a wavelength in a range of 400-500 nm. In at least an embodiment, the light source 1010 may emit light having a wavelength in a range of 400-410 nm, in a range of 404-406 nm, or having a wavelength of approximately 405 nm. Alternatively, light source 1010 may emit light having a wavelength in a range of 465-475 nm, in a range of 469-471 nm, or having a wavelength of approximately 470 nm for a lower cost alternative to the 405 nm light sources. Additionally, as noted above, it will be understood that the light source 1010 may include a light source that emits only light having a wavelength of in the range of 400-500 nm, only light having a wavelength of in the range of 400-410 nm, only light having a wavelength of in the range of 465-475 nm, only light having a wavelength of in the range of 404-406 nm, only light having a wavelength of in the range of 469-471 nm, only light having a wavelength of approximately 405 nm, or only light having a wavelength of approximately 470 nm.

Light source 1010 may be provided inside of main body 1000, and main body 1000 can be formed of a transparent material. Alternatively, light source 1010 may be provided on an exterior surface of main body 1010. Additionally, light source 1010 may include a plurality of light sources. For example, as seen in FIG. 18, a device may have a transparent main body 1100 with multiple light sources 1110 provide therein.

Control electronics 1030 may be structured to control supply of power from power source 1020 to light source 1010. Control electronics 1030 can control the light source 1010 to turn on for a set period of time. Additionally, control electronics 1030 can cause light sources 1010 to turn on and off at a predetermined frequency and duty ratio. The flickering of light sources 1010 can enhance the user experience to show that the device is working.

Control electronics 1030 may be controlled by user interface 1040. User interface 1040 may take the form of a pressure sensor, dial, knob, button, switcher, slider, or any other suitable structure. User interface 1040 may be used by the user to control the activation time of the device, modes of the device, frequency or duty ratio of the flickering light, or other functions. The control electronics 1030 may serve to activate indicator lights, sound, vibration or other sensory stimulus to remind a user when to use the device. Additionally, the control electronics may include communication circuits to allow the device to link with smart phones or other devices, which could allow the user to track use of the device for pathogen inactivation or set reminders of when to use the device, such as prior to meal times, before or after leaving work, during children activities, etc.

It will be understood that an important benefit of the handheld devices described above is their portability. The devices can be easily used in the home, in the car/bus/train/plane, at work, at restaurants, in the gym, and anywhere else a user may go. The handheld devices may also be particularly useful for outdoors activities, such as camping, hiking, boating, fishing, hunting, etc., where a user may be exposed to a variety of pathogens, but does not have ready access to clean water and soap.

It will also be understood that main body 1000 can take a variety of forms. For example, in one embodiment, such as shown by main body 1100 in FIG. 18, the main body may be formed in the approximate size and shape as a bar of soap. This will reinforce the function of the device to the user, for example, but encouraging the user to rub the device over their hands as they would a bar of soap when pathogen inactivation on the hands is desired. Alternatively, an embodiment of the device could be realized in the cover or body of a cell phone, for example, allowing for inactivation of pathogens without having to carry an alternative device. Additionally, an embodiment of the device could be realized in the body of a brush, which could then be used for brushing pets or other animals to inactivate pathogens on their skin during grooming. Generally, transparent accoutrements where pathogens reside and be transferred from the surface to hands, food or water, can be configured to accommodate sanitation capabilities.

It will also be understood that an embodiment of the device can be made so that an outer surface is waterproof. Thus, the handheld device could be used under running water in lieu of traditional soap. Additionally, a waterproof handheld device could be used in dental applications, by being incorporated into a toothbrush or other dental appliance to help supplement traditional brushing in flossing to inactivate the pathogens that cause halitosis and gingivitis.

As discussed above, the amount of pathogens inactivated by visible light will vary with the power of the light and the length of exposure. In at least one embodiment of the handheld device, the goal is to achieve at least 90% inactivation of pathogens, which is similar to the efficacy of store-bought commercial hand cleansers based on common usage patterns

In a study described below, it was determined that a total dose of 900 mJ/cm2 is sufficient to inactivate approximately 90% of a bacterial pathogen. Thus, if it is desired for the handheld device to achieve 90% inactivation in 5 seconds of use, the handheld device will need to provide an irradiance of 180 mW/cm2. Alternatively, if 90% inactivation is desired in 10 seconds of use, an irradiance of 90 mW/cm2 will be necessary.

The power of the light source 1010 in the handheld device will depend on the desired inactivation time and the geometry of the device. For example, if the handheld device is a sphere with radius of 4 cm, having a light source at the center, and 10 second inactivation (i.e., irradiance of 90 mW/cm2) is desired, then the light source will need to emit approximately 18.1 W of light. In more complicated geometries, it will be understood that it will be more difficult to achieve a uniform irradiance at an outside surface of the handheld device. Accordingly, given that a user will be rubbing the device back and forth in their hands or over an object, one can consider an average irradiance at an outer surface of the device.

In development of the embodiments described above, the following study was conducted regarding the efficacy of visible light to inactivate pathogens.

A challenge suspension of Staphylococcus aureus containing approximately 109 CFU/mL was prepared in 0.9% Sodium Chloride Irrigation, USP. A total of eight sterile stainless steel coupons 3 inches×3 inches in size were each contaminated with a 0.1 mL aliquot of the challenge suspension and dried at 35 degrees C. for approximately 15 minutes. Six of the contaminated coupons were individually exposed within an antimicrobial light box for five minutes. Each coupon was maintained in a horizontal position, contaminated-side up, during the exposure period. Three of the six coupons were exposed at a distance of approximately 3 inches below the upper bulbs. Inside the light box, the coupons were exposed to 405 nm light at an approximate irradiance of 3 mW/cm2. Following exposure, the viable microbial population remaining on each coupon was determined by rinsing, diluting, and plating aliquots, in duplicate. Two contaminated coupons were not exposed to the antimicrobial light box and were also evaluated for viable microbial population. These coupons served as untreated baseline controls.

The following tables summarize the results of the study.

TABLE 1
Baseline microbial Recoveries (Untreated)

| | | | Mean Log₁₀ |
|---|---|---|---|
| Test description | CFU/coupon | Log₁₀ [CFU/coupon] | CFU/Coupon |
| Baseline (untreated) | 3.9750 × 10⁸ | 8.5993 | 8.6087 |
| Coupon #1 | | | |
| Baseline (untreated) | 4.150 × 10⁸ | 8.6180 | |
| Coupon #2 | | | |

TABLE 2
Post exposure microbial recoveries
Antimicrobial light box - 5 minute exposure

| | | | | Mean Log₁₀ |
|---|---|---|---|---|
| | | Log₁₀ | Mean Log₁₀ | Reduction from |
| Test Description | CFU/Coupon | [CFU/coupon] | [CFU/coupon] | baseline coupons |
| Treated Coupon #1 | 4.5250 × 10⁷ | 7.6556 | 7.6869 | 0.9218 |
| Treated Coupon #2 | 5.5250 × 10⁷ | 7.7423 | | |
| Treated Coupon #3 | 4.60 × 10⁷ | 7.6628 | | |
| Treated Coupon #4 | 1.4425 × 10⁷ | 7.1591 | 7.4585 | 1.1502 |
| Treated Coupon #5 | 5.6750 × 10⁷ | 7.7540 | | |
| Treated Coupon #6 | 2.90 × 10⁷ | 7.4624 | | |

In the table above, treated coupons #1-#3 were placed approximately 1 cm from the light source, and treated coupons #4-#6 were placed approximately 3 inches from the light source. The tables above show that the 5 minute exposure of light was successful in reducing the number of pathogens by approximately a factor of 10, i.e., a 90% reduction.

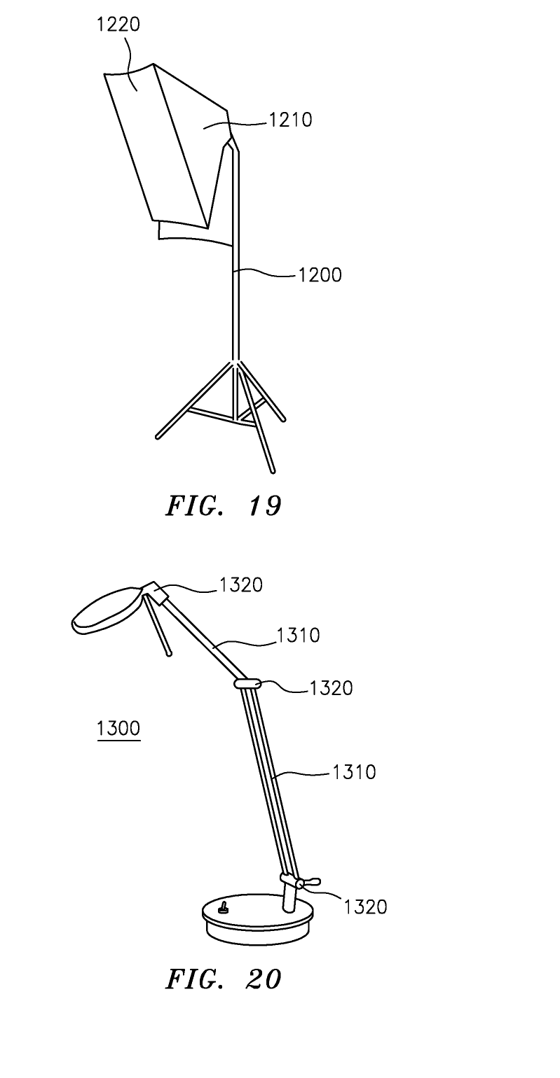

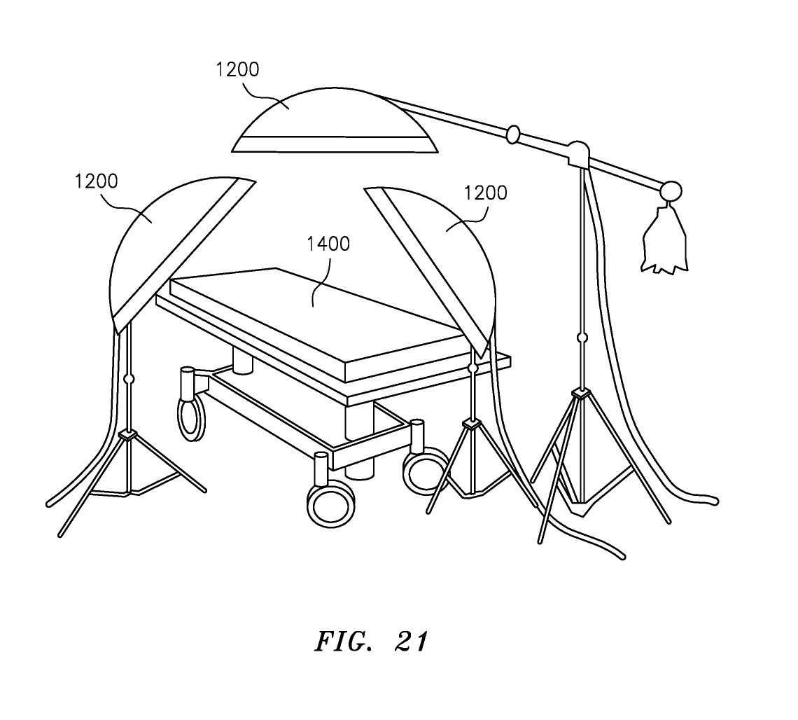

FIGS. 19-21 show an embodiment of a device and method for inactivating pathogens on a surface. For example, FIG. 19 shows a device 1200 having a hood 1220 and a light source 1210 provided within hood 1210. In FIG. 19, the light source 1210 is not directly shown, but the reference numeral 1210 indicates the approximate position where the light source is located inside of hood 1220. Hood 1210 can be internally reflective and structured to direct the light at a surface where pathogen inactivation is desired. FIG. 20 shows another embodiment in which a light source can be provided in a structure 1300 having articulated arms 1310 and joints 1320, to aid in directing the light exactly where it is desired.

An embodiment of the hood 1210 may be realized by an unfurling mechanism similar to an umbrella. Inside surfaces of the hood 1210 could be coated or formed of a reflective material, to help ensure that as much light as possible is directed to the target surface. Additionally, reflectors can be provided behind the light source for the same purpose of directing as much light as possible to the target surface.

In at least an embodiment, light source 1210 may emit light having a wavelength in the range of 400-500 nm, light having a wavelength in a range of 400-410 nm, or light having a wavelength of approximately 405 nm.

Additionally, as noted above, it will be understood that the light source 1210 may include a light source that emits only light having a wavelength of in the range of 400-500 nm, only light having a wavelength of in the range of 400-410 nm, or only light having a wavelength of approximately 405 nm.

The devices shown in FIGS. 19 and 20 can be used by first positioning the light source a predetermined distance from the surface for which pathogen inactivation is desired. The predetermined distance depends on the geometry of the light source, any hood, and the desired area of inactivation. For example, for a desk-sized version of the device, it may be determined that the device will have an inactivation area of 1000 cm2 when positioned 30 cm away. However, it will be understood that the device is not limited to this arrangement, and it will be understood that a wide variety of geometries and distances will be encompassed by the method being described.

Once the light is positioned appropriately, it can be aimed so that the light is directed to the area where pathogen inactivation is desired. Because the device is emitting light having a wavelength of 400-500 nm, this falls within the visible light spectrum and is not dangerous to vision or skin. Therefore, a user could turn on the light source 1210 while aiming the device so that an illuminated area will be shown to aid in aiming.

Next, the device will be activated for a predetermined amount of time. As described above, the predetermined time depends on the power of the light source 1210 and the level of pathogen activation desired. Examples above have been described for achieving various levels of pathogen inactivation at varying levels of exposure time. However, it will be understood that longer or shorter activation times are possible by varying the power of the light source, and that these are encompassed within the scope of the device and method described herein.

Present FIG. 21 shows at least one embodiment of how devices 1200 may be used. For example, one or more devices 1200 may be provided around an operating table, and be continuously turned on to provide persistent pathogen inactivation of the surgical field during an operation. Alternatively, at least an embodiment of the device could also be realized in the form of a light “faucet” or light “shower” to be used, for example, for surface pathogen inactivation of one's hands or body after working in a contaminated environment without requiring the use of water, which could be useful in locations where water supplies are scarce. Additionally, at least an embodiment of the device could be implemented in conjunction with traditional water showerheads and faucets, providing supplemental pathogen inactivation due to the light exposure at the same time as the hand washing or showering. Additionally, an embodiment of the device can be used for persistent inactivation of pathogens of a works surface such as a food preparation area or a laboratory workspace.

The embodiments described above have a number of advantages over conventional methods of surface pathogen inactivation. For example, the devices and methods above achieve a much higher level of pathogen inactivation than conventional visible light pathogen inactivation techniques in a much shorter time. Additionally, as compared with traditional methods of soap-and-water or alcohol rub pathogen inactivation, the embodiments described above will result in less skin irritation while providing a more uniform pathogen inactivation of hands and other surfaces. Additionally, because the embodiments described above use visible light, there is no danger to vision or skin. In fact, the use of 405 nm light may have anti-aging and anti-wrinkle properties.

The benefits from using the embodiments described above should result in more consistent pathogen inactivation among healthcare workers, food service workers, students, etc, thereby realizing a significant public health benefit.

It will also be understood that the 405 nm light described above is not as damaging to plastics as is other ultraviolet light used for pathogen inactivation. Thus, these embodiments may be useful for pathogen inactivation on instruments, tools, or surfaces that are sensitive to ultraviolet light.

Additionally, the handheld embodiments described above provide a convenient way for consumers to experience similar benefits of surface pathogen inactivation in a portable form, without experiencing the negative skin effects of traditional hand rubs.

In addition to the device described above, it may be desirable to have a system that utilizes both visible light and UV light for continuous pathogen inactivation. For example, the device described above is useful for inactivating pathogens on hands or other small objects that can be inserted into the device. However, there may be situations where continuous pathogen inactivation may be desired on a surface that is impractical for insertion into a device. For example, in the food service industry, prep areas such as counters or service areas such as salad bars are constantly exposed to a variety of pathogens throughout the day, as well as food that is left out on a salad bar or buffet. Additionally, work stations or counters in labs or research field stations may be exposed to pathogens. Medical or veterinarian facilities may wish to have continuous pathogen inactivation on examination tables or operating theaters. Even mundane surfaces with high frequency human contact could benefit from pathogen inactivation, such as doorknobs, ATM keypads, restroom fixtures, etc. It is beneficial to have a system that utilizes both visible light and UV light because of possible safety issues associated with UV light exposure. For example, when no people are present, both the visible light and the UV light can be used in combination to enhance efficacy. In contrast, when a person is present, the UV light can be turned off or set to a reduced intensity so as not to exceed predetermined safety thresholds. Additionally, it is possible that a pathogen may develop resistance to a particular wavelength of light. Accordingly, providing a system that uses multiple wavelengths for inactivation provides redundancy as a safeguard against resistant pathogens. A system for pathogen activation based on these concepts is discussed below.

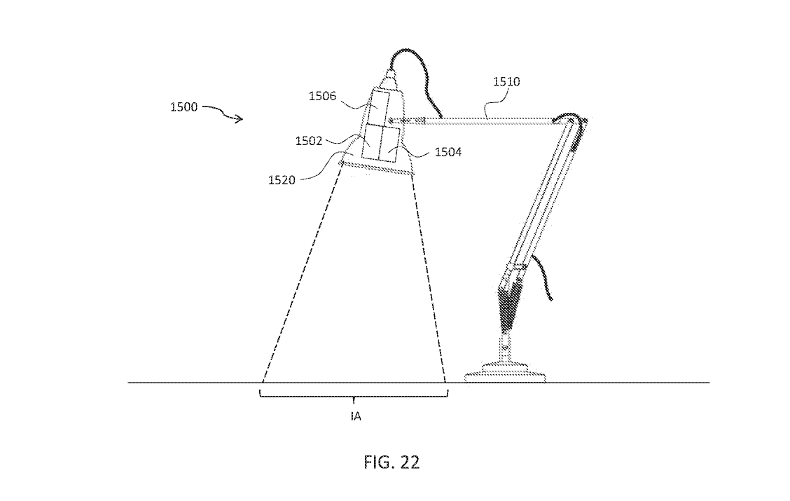

FIG. 22 shows an embodiment of a system 1500 for directing light to a surface for inactivating pathogens within an inactivation area IA. The inactivation area IA is an area within which a user desires inactivation of pathogens. Even though inactivation area IA appears one-dimensional because of the planar nature of FIG. 22, it will be understood that inactivation area IA is actually two-dimensional.

The system 1500 may include a first light source 1502 that emits first light having a peak wavelength in a range of 400 nm to 500 nm. Alternatively, first light source 1502 may emit first light having a spectrum with a peak wavelength in a range of 400 nm to 410 nm or in a range of 450 nm to 470 nm. In at least an embodiment, a peak wavelength of the first light is approximately 405 nm. Additionally, in at least an embodiment, the first light may have a spectrum with a full width at half maximum height of approximately 10 nm or less. In other words, the first light source 1502 may emit visible blue light that has pathogen inactivation properties as described above.

Additionally, the system may include a second light source 1504 that emits second light having a spectrum with a peak wavelength in a range of 185 nm to 400 nm. In at least an embodiment, the spectrum of the second light may have a peak wavelength in a range of 240 nm to 280 nm. In at least an embodiment, the second light may have a spectrum with a peak wavelength of approximately 254 nm, 260 nm, or 275 nm. Additionally, in at least an embodiment, the second light may have a spectrum with a full width at half maximum height of approximately 10 nm or less. In other words, the second light source 1504 may emit UV light.

In at least an embodiment, the inactivation area IA of the system may have an area of 10 square feet or less. However, it will be understood that the area is not limited to this value, and other sized areas may be used as well. In at least another embodiment, such as used with a salad bar, buffet table, or countertop, the inactivation area may have a significant length, but it will have a width of 4 feet or less. However, it will be understood that the area is not limited to this value, and other sized areas may be used as well. It will also be understood that an illumination area of the first light and an illumination area of the second light are limited to be substantially within the inactivation area. This insures that as much power as possible is being directed towards the desired inactivation area. It will also be understood that the inactivation area IA may be any variety of shape as required by the particular application. For example, inactivation area IA may be square, rectilinear, polygonal, circular, elliptical, or any other two-dimensional shape. It will also be understood that the inactivation area is not required to be planar. For example, the light used for pathogen inactivation can track the contours of a three-dimensional surface, such as lab equipment, serving trays/bowls, food items on display, human body parts during medical procedures, or any other three-dimensional surface that requires pathogen inactivation.

The system may also include control electronics 1506 that are operably connected with the first light source 1502 and the second light source 1504. The control electronics 1506 are configured to control a first power state of the first light source 1502 and a second power state of the second light source 1504.

For example, in at least an embodiment, the first power state may be switchable between an ON state and an OFF state, and the second power state may be switchable between an ON state and an OFF state. The first power state and the second power state may be controlled independently of each other. For example, the first power state may be set to the ON state while the second power state may be set to the OFF state, or vice versa. It will also be understood that in addition to binary ON/OFF states, the first power state and the second power state may also include a range of power. For example, the first power state and the second power state may be independently set at anywhere from 0% to 100% of full power.

The first light source 1502 and the second light source 1504 may take a number of forms. For example, the first light source 1502 and the second light source 1504 may be configured out of low pressure lamps, high pressure lamps, cold-cathode lamps, arrays of LEDs, or other sources of light capable of emitting light of the desired spectrum.

Additionally, it will be understood that the physical arrangement, size, and/or shape of the first light source 1502, the second light source 1504, and the control electronics 1506 are not limited to what is shown in FIG. 22. Instead, FIG. 22 merely shows the first light source 1502, the second light source 1504, and the control electronics 1506 as a block diagram, and any suitable arrangement of these structures is possible.

There are a number of different configurations in which the first light source 1502 and the second light source 1504 may be provided. For example, as shown in FIG. 22, the first light source 1502 and the second light source 1504 may be placed on the end of an articulated arm 1510. A light guide such as hood 1520 can be provided to direct as much light as possible from the first light source 1502 and the second light source 1504 to the target inactivation area. The light guide may have a reflective inner surface so that as much energy as possible is transmitted from the light sources to the inactivation area. It will be understood that the light guide is not limited to the hood 1520 shown in FIG. 22. Other types of light guides such as collimators, mirrors, canopies, or other suitable structures may be used to shape and direct the emitted light. The articulation of articulated arm 1510 allows for the light to be aimed at whatever surface requires inactivation of pathogens.

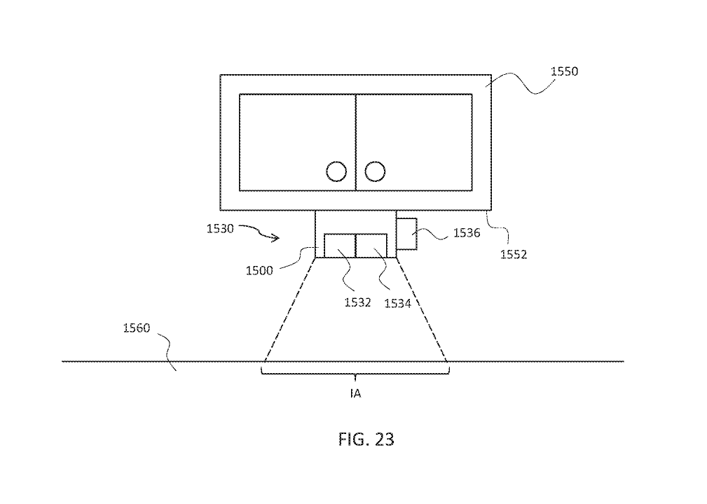

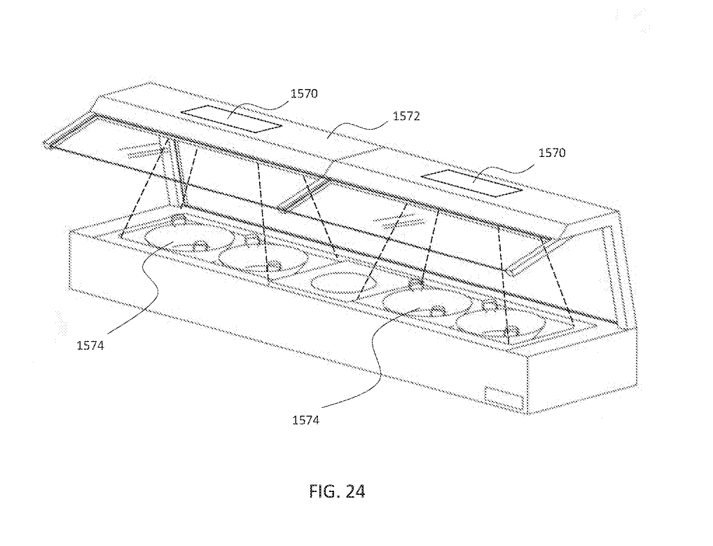

As another embodiment, as shown in FIG. 23, the system 1530 may include first light source 1532, second light source 1534, and control electronics 1536 provided in a light fixture 1540 provided on a surface and aimed at the work area for which pathogen inactivation is desired. For example, as seen in FIG. 23, the light fixture 1540 could be fixed to the lower surface 1552 of a cabinet 1550 above a work table 1560. Alternatively, as seen in FIG. 24, systems 1570 could be provided on a panel 1572 of a salad bar or food display case and aimed to emit light at the food display area 1574. Because the systems 1570 include a first light source and a second light source as described above, there are a number of ways the systems 1570 could be used to inactivate pathogens. For example, during non-business hours when nobody is around, the systems 1570 could be controlled to emit both light with a peak wavelength in the range of 240 nm to 280 nm (i.e., UV light) and light with a peak wavelength in the range of 400 nm to 410 nm (i.e., germicidal blue light) During business hours when people are present, systems 1570 could be controlled to emit just the germicidal blue light, or the germicidal blue light with lower levels of UV light so as not to exceed safe exposure levels of UV light.

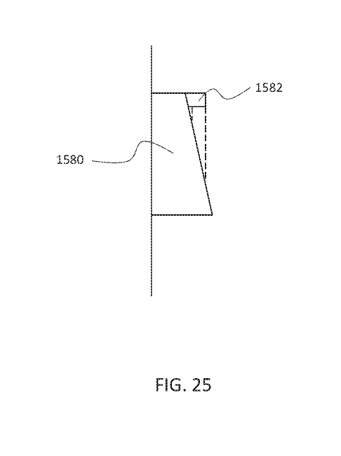

Many other applications of the system are possible. For example, FIG. 25 shows an embodiment in which a system 1582 is attached to a public terminal 1580 such as an ATM machine or ticket dispenser. Additionally, the pathogen inactivation system described above may be used to inactivate pathogens on lab tables, food preparation areas, food display areas including food, medical facilities such as examination tables or operating theaters, lavatory fixtures, door handles, or any other surface where humans may encounter pathogens.

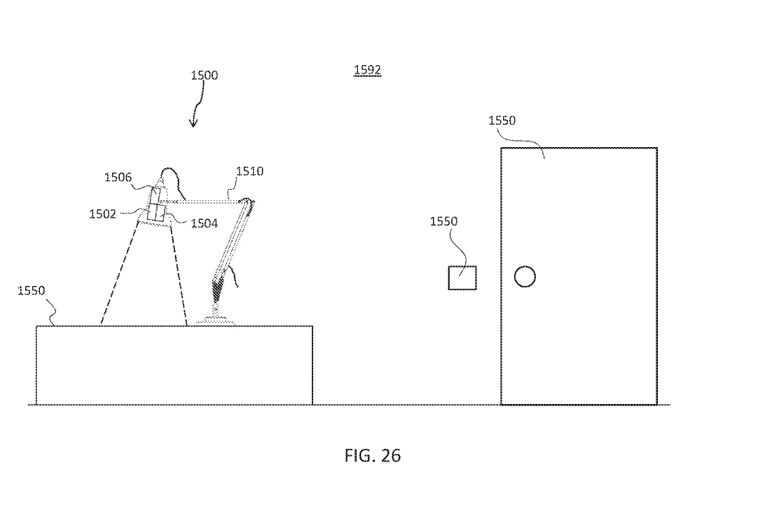

Because exposure to UV light may have adverse effects on human skin and eyes, it is helpful to provide safeguards that will protect users of the system from inadvertent exposure to UV light. For example, FIG. 26 shows an embodiment in which the pathogen inactivation system 1500 may be used in a room 1592 that has a countertop workspace 1508, such as a lab or food preparation area. In this embodiment, the system 1500 is shown using a first light source 1502 and a second light source 1504 provided on an articulated arm 1510. Additionally, the system may include a detector 1590 operably connected to the control electronics and configured to detect whether a person is present in the room 1592 with system 1500. The detector may be operably connected to the control electronics via a wired connection or via a wireless connection.

The detector 1590 may take any of a number of possible forms. For example, the detector 1590 could be a motion detector configured to detect motion of a person within the room 1592. Alternatively, the detector 1590 could be a sound detector that detects voices or other sounds associated with a person being in the room 1592. Alternatively the detector 1590 could be a thermal detector that detects a person in the room 1592 via body heat. Alternatively the detector 1590 could be a magnetic detector configured to detect opening and shutting of the door 1594.

In operation, the detector 1590 will send a signal to the control electronics 1506 of the system 1500, and the control electronics 1506 will be configured to determine whether a person is present in the room 1592. If the control electronics 1506 determine, in response to the signal from the detector 1590, that a person is present in the room 1592, then the second power state of the second light source 1504 will be adjusted. For example, the control electronics 1506 may set the second power state to an OFF state. Alternatively, the control electronics 1506 may set the second power state to a reduced power state such that the amount of UV light to which the user is exposed is with in predetermined safety limits. Setting the second power state to a reduced power state would protect users from UV exposure while still allowing for UV light from the second light source 1504 to augment the pathogen inactivation of the visible light from the first light source 1502.

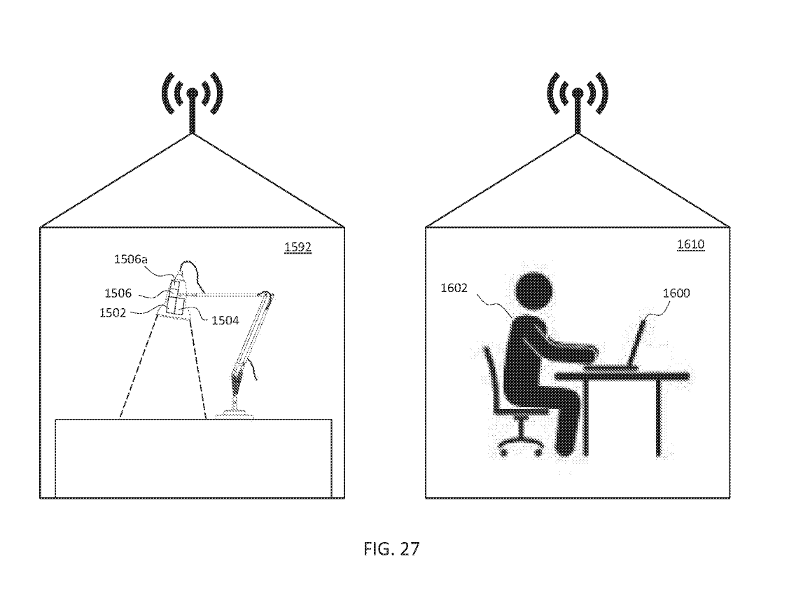

Additionally, in another embodiment shown in FIG. 27, the control electronics 1506 may include communication electronics 1506a to allow for communication with a remote terminal 1600 provide at a location 1610 remote from room 1592. The remote terminal 1600 may be a personal computer, a cell phone, a tablet, or other suitable device. The remote terminal 1600 may communicate with the communication electronics 1506a of the control electronics 1506 via a local wired or wireless network, via the internet, via radio waves, or by another suitable method of communication. The remote terminal 1600 may be configured so that a user 1602 can determine the first power state and the second power state from outside the room 1592, i.e., without having to be physically present in the room 1592. This provides a layer of safety by allowing the user 1602 to confirm that pathogen inactivation is occurring without exposing the user 1602 to UV light. Additionally, the user 1602 may be able to use the remote terminal 1600 to communicate with the control electronics 1506 via communications electronics 1506a to set the first power state and second power state to a different setting from a position outside of the room 1592.

As a safety measure in an alternative embodiment, the control electronics 1506 may also be configured to set the second power state to an ON state or an increased power state after a predetermined first period of time following an input from the user. For example, the user could set the control electronics 1506 to set the second power state to an ON state (i.e., turn on the second light source 1504) five minutes after a confirmation command. After issuing the confirmation command, the user could then leave the room. The inactivation area IA will be illuminated with UV light five minutes later when the second power state is set to ON.

Additionally, the control electronics 1506 may be configured to set the second power state to an OFF state after being in an ON state for a predetermined second amount of time. For example, in an embodiment, it may be determined that, based on power levels of the second light source 1504 and distance from an inactivation area IA, four hours of illumination from the second light source is sufficient to achieve a predetermined level of pathogen inactivation on the inactivation area IA. Thus, the control electronics 1506 could be configured to set the second power state to an OFF state after being in an ON state for four hours. This will reduce power consumption as well as help to protect against inadvertent exposure to UV light.

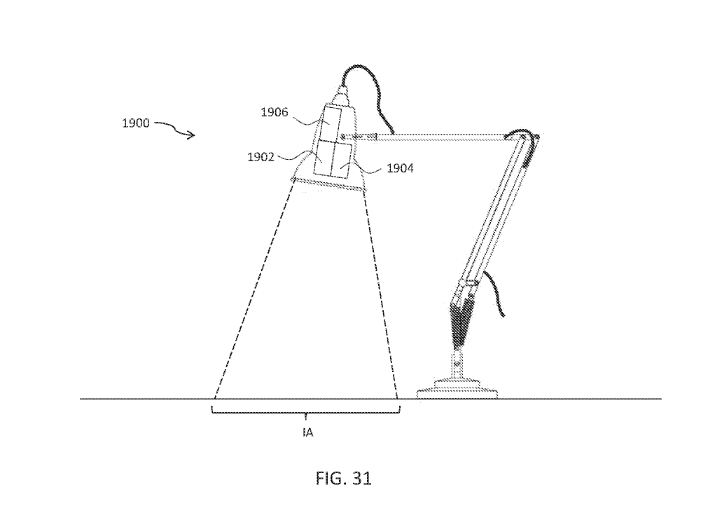

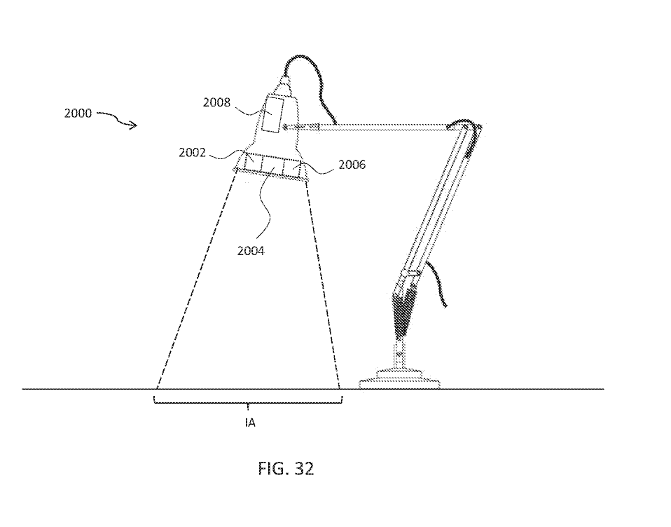

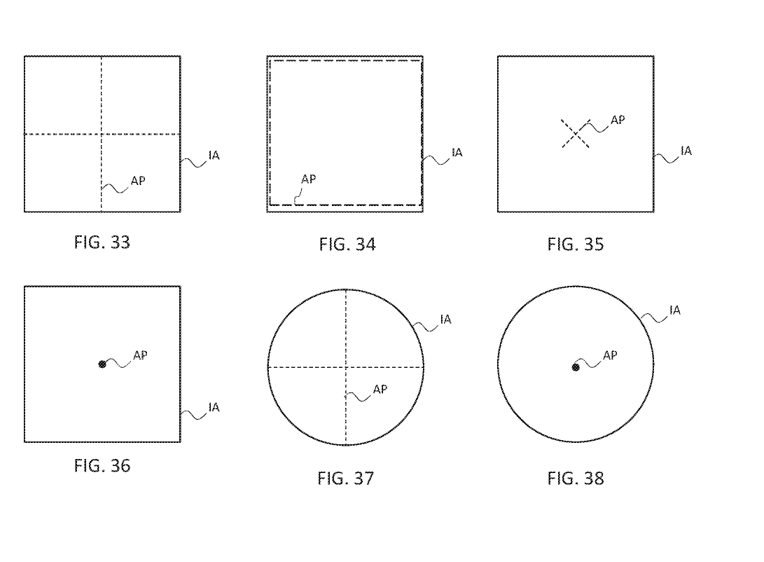

Additionally, as an aid in aiming the pathogen inactivation system to insure adequate coverage of the desired work area, the control electronics may be configured to set the system 1500 into an aiming state in which the second light source 1504 is turned off and the first light source 1502 is set to emit light in a pattern that indicates the area to be illuminated. In a simple configuration, the aiming state may simply be turning off the second light source and turning on the first light source, and using the visible light from the first light source as a guide to determine what area is going to be illuminated. For example, the first light source may illuminate a circular or rectilinear region in visible light, and the user would know that the UV illumination will illuminate the same region when turned on. In another embodiment, the first light source and the second light source may be arrays of LEDs. It may be necessary in some applications to center the illumination on a particular spot. Thus, in the aiming state, the LEDs of the first light source could be selectively turned on and/or shaped by a collimator to create an X pattern or a crosshairs pattern of light that could be used to locate a center of the illumination area. It will be understood that the specific pattern in the aiming state is not limited to these examples, and other patterns may be used depending on the specific application. As an example, FIGS. 32-37 show a series of at least some possible inactivation areas IA and aiming patterns AP formed by the first light.

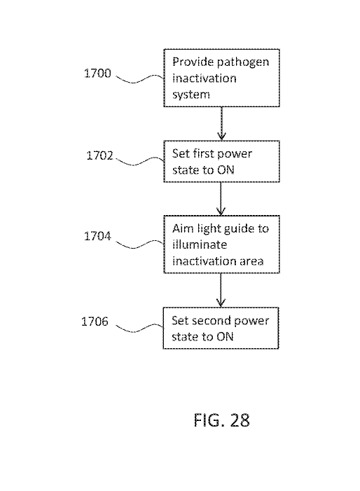

Present FIG. 28 shows a flowchart describing an embodiment of a method for inactivating pathogens using the system described above. For example, block 1700 illustrates a step of providing a pathogen inactivation system. The pathogen inactivation system of block 1700 may include a first light source that emits light having a peak wavelength in the range of 400 nm to 410 nm, a second light source that emits light having a peak wavelength in the range of 240 nm to 280 nm, and a light guide structured to direct light from the first light source and light from the second light source to a target area. Block 1702 illustrates a step of setting a first power state of the first light source to an ON state. Block 1704 illustrates a step of aiming the light guide so that light from the first light source illuminates an area where inactivation of pathogens is desired. Block 1706 illustrate a step of setting the second power state of the second light source to an on state.

The method may also include a step of continuously detecting with a detector whether a person is present in the room. For example, block 1708 illustrates a determination of whether a person is in the room. If yes, then the method proceeds to block 1710 in which the second power state is set to an OFF state. If no, then the method loops back to block 1708 to continuously determine whether a person is in the room.

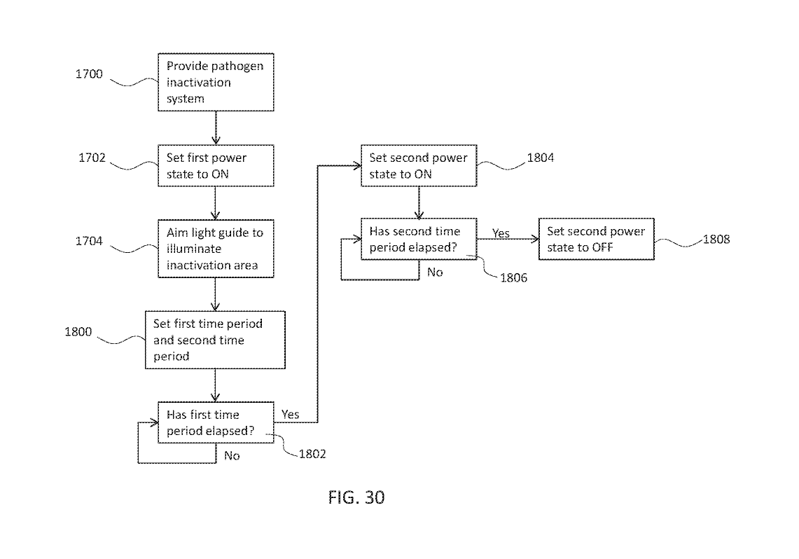

In at least another possible embodiment of the method shown in FIG. 30, after aiming the light guide, block 1800 illustrates a step of setting a first time period. In block 1802, it is continuously determined whether the first time period has elapsed. If no, then the method loops back to block 1802 to monitor whether the time period has elapsed. If yes, then in block 1804 the second power state is set to an ON state. The method may also include setting a second period of time in block 1800. Once the second power state is set to ON in block 1804, it is determined in block 1806 whether the second time period 1806 has elapsed. If no, the method loops back to block 1806 to continue monitoring the second time period. If yes, then in block 1808 the second power state is set to OFF.

It will be also understood that the system may use only one of UV light and germicidal blue light to inactivate pathogens, and use a visible light source as an aiming guide.

For example, as seen in FIG. 31, the system 1900 may include a first light source 1902 and a second light source 1904. The first light source 1902 may emit first light having a peak wavelength in the range of 185 nm to 500 nm. In at least an embodiment, the first light may have a peak wavelength in a range of 240 nm to 280 nm, in a range of 400 nm to 500 nm, in a range of 400 nm to 410 nm, or in a range of 450 nm to 470 nm. The second light source 1904 may emit second light that is visible light. The system 1900 may further include control electronics 1906 configured to control a first power state of the first light source 1902 and a second power state of the second light source 1904. The first power state and the second power state may be independently controlled between an ON state, an OFF state, or an intermediate state where the respective light source is supplied with less than full power. The second light source 1904 may be configured to emit light in a predetermined pattern that indicates the inactivation area to be illuminated. For example, the second light source 1904 may illuminate a round or rectilinear area that shows where the germicidal light will be illuminated. Alternatively, the second light source may be further collimated and/or shaped to project light in a shape indicating a center of the inactivation area, such as an X-shape or cross shape. Alternatively, the second light source 1904 may be an array of LEDs arranged in a predetermined pattern to project an aiming pattern. It will be further understood that an illumination area of the first light may be limited to be substantially within the inactivation area. With this system, the user can use the second light as an aiming guide to determine where the first light will be illuminated.

In the embodiment of FIG. 31, the first light and the second light may have a spectrum having a full width at half maximum of approximately 10 nm or less. The second light may have a spectrum having a peak wavelength in the visible light spectrum. Further, the second light may have a spectrum having a peak wavelength in a range of 400 nm to 410 nm or in a range of 450 nm to 470 nm.

FIG. 32 shows at least another embodiment in which a system 2000 may include three light sources. A first light source emits 2002 first light having a spectrum with a peak wavelength in a range of 185 nm to 400 nm. In at least an embodiment, the first light source may emit light having a peak wavelength in a range of 240 nm to 280 nm. A second light source 2004 emits second light having a spectrum with a peak wavelength within the visible light spectrum. Similar to the embodiment of FIG. 31, the second light source may be configured may be configured to emit light in a predetermined pattern that indicates the inactivation area to be illuminated as an aiming guide. A third light source 2006 may emit third light having a spectrum with a peak wavelength in a range of 400 nm to 500 nm, a range of 400 nm to 410 nm, or a range of 450 nm to 470 nm. An illumination area of the first light and the third light may be limited to be substantially within the desired inactivation area IA. The system of FIG. 31 further includes control electronics 2008 configured to control a first power state of the first light source 2002, a second power state of the second light source 2004, and a third power state of the third light source 2006, and the first, second, and third power states may be independently controlled. With this system 2000, a user can have the benefits of two different mechanisms of pathogen inactivation (i.e., the UV first light and the germicidal blue third light), while using the second light as a convenient visual guide for aiming the first light and the third light.

It will also be understood that there are some medical treatments that use certain frequencies of UV light from 300 nm to 400 nm, such as UV therapy for treating psoriasis. Additionally, there are some therapeutic uses of 405 nm light, such as for acne treatments. Any of the embodiments above may be adapted so that germicidal light can be provided at the same time as the therapeutic light. For example, referring to the embodiment of FIG. 22, in at least an embodiment, the first light source 1502 may emit light having a peak wavelength in a range of 300 nm to 400 nm (i.e., therapeutic UV light) or may emit light having a peak wavelength of approximately 405 nm (i.e., therapeutic blue light). Additionally, the second light source 1504 may emit light having a peak wavelength in a range of 185 nm to 300 nm (germicidal UV light) or in a range of 400 nm to 500 nm (germicidal blue light). Thus, the treatment site could be simultaneously provided with pathogen inactivation to reduce the risk of infections during the treatment.

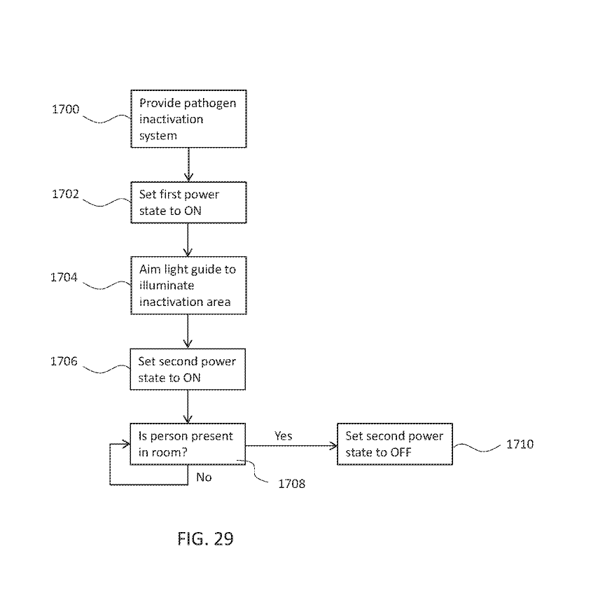

It was described above that a remote terminal may be used to control the pathogen inactivation system. The remote terminal may be used with a non-transitory computer readable medium that includes computer readable instructions that, when executed by the remote terminal, cause the system to perform the methods illustrated in FIGS. 28 through 30. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A system for inactivation of pathogens within an inactivation area on a surface, the system comprising:
a first light source that emits first light having a peak wavelength in a range of 400 nm to 500 nm;
a second light source that second emits light having a peak wavelength in a range of 185 nm to 400 nm; and
control electronics configured to control a first power state of the first light source and a second power state of the second light source;
wherein the first power state and the second power state are independently controlled;
wherein an illumination area of the first light and an illumination area of the second light is limited to the inactivation area.

2. The system of claim 1, further comprising a light guide structured to direct the first light and the second light to the inactivation area.

3. The system of claim 1, wherein the first light source and the second light source are provided on an articulated arm.

4. The system of claim 1, wherein the first light source and the second light source are provided on an underside of a first surface provided above a work area.

5. The system of claim 1, wherein the first light source and the second light source are provided in a room;
the system further comprises a detector configured to detect a presence of a person in the room; and
the control electronics are configured such that the second power state of the second light source is set to an off state in response to a person being detected in the room.

6. The system of claim 1, wherein the first light source and the second light source are provided in a room;
the system further comprises communication electronics operably connected to the control electronics; and
the communications electronics the control electronics are configured such that a user can determine the second power state from a location outside of the room; and
the communications electronics and the control electronics are configured such that the user can control the second power state from the location outside of the room.

7. The system of claim 1, wherein the control electronics are configured such that, in response to an input from a user, the second power state is set to an on state after a first period of time has elapsed.

8. The system of claim 7, wherein the control electronics are configured such that the second power state is set from the on state to an off state after a second period of time has elapsed.

9. A method of inactivating pathogens within an inactivation area on a surface, the method comprising:
providing a system comprising:
a first light source that emits first light having a peak wavelength in a range of 400 nm to 500 nm;
a second light source that emits second light having a peak wavelength in a range of 185 nm to 400 nm;
a light guide structured to direct the first light and the second light to the inactivation area;
setting a first power state of the first light source to an on state;
aiming the light guide so that the first light illuminates the inactivation area; and
setting the second power state of the second light source to an on state;
wherein an illumination area of the first light and an illumination area of the second light are limited to the inactivation area.

10. The method of claim 9, wherein the first light source and the second light source are provided in a room; and
the method further comprises:
continuously detecting with a detector whether a person is present in the room; and
setting the second power state to an off state in response to detection of a person in the room.

11. The method of claim 9, wherein the setting the second power state of the second light source to an on state comprises:
setting a first period of time; and
setting the second power state to the on state after the first period of time has elapsed.

12. The method of claim 11, further comprising:
setting a second period of time; and
setting the second power state from the on state to an off state after a second period of time has elapsed.

13. A system for inactivation of pathogens on a surface, the system comprising:
a first light source that emits first light having a peak wavelength in a range of 185 nm to 500 nm;
a second light source that emits second light that is visible light; and
control electronics configured to control a first power state of the first light source and a second power state of the second light source;
wherein the first power state and the second power state are independently controlled;
wherein the second light source is configured to emit light in a predetermined pattern that indicates an inactivation area to be illuminated;
wherein the system is configured such that an illumination area of the first light is limited to the inactivation area.

14. The system of claim 13, wherein the second light has a peak wavelength in the visible spectrum and a full width at half maximum of approximately 10 nm.

15. The system of claim 13, wherein the peak wavelength of the second light is in a range of 400 nm to 500 nm.

16. The system of claim 13, further comprising:
a third light source that emits third light that is visible light;
wherein a peak wavelength of the first light is in a range of 185 nm to 400 nm and a peak wavelength of the third light is in a range of 400 nm to 500 nm;
the control electronics are further configured to control a third power state of the third light source;
the first power state, the second power state, and the third power state are independently controlled; and
the system is configured such that an illumination area of the third light is limited to the inactivation area.

17. The system of claim 13, wherein the inactivation area has an area of 10 square feet or less.

18. The system of claim 13, wherein the inactivation area has a width of 4 feet or less.

19. The system of claim 1, wherein the inactivation area has an area of 10 square feet or less.

20. The system of claim 1, wherein the inactivation area has a width of 4 feet or less.
